(19) United States
(12) Patent Application Publication
(10) Pub. No.: US 2025/0253037 A1
(43) Pub. Date: Aug. 7, 2025

(54) SYSTEMS AND METHODS FOR AUTOMATED CODE CLASSIFICATION AND NATURAL LANGUAGE GENERATION

(71) Applicant: DECISION DOC INC., Sheridon, WY (US)

(72) Inventors: Heriberto Paredes, Sugar City, ID (US); Tom Emil Langeland, Solana Beach, CA (US)

(21) Appl. No.: 19/044,946

(22) Filed: Feb. 4, 2025

Related U.S. Application Data
(60) Provisional application No. 63/549,841, filed on Feb. 5, 2024.

(51) Int. Cl. G16H 40/20; G16H 20/00

(52) U.S. Cl. CPC G16H 40/20; G16H 20/00

(57) ABSTRACT

Methods and systems for next-action recommendation and support interface generation are disclosed. A set of intake data is received and at least one support-based evidenced code is generated based on the set of intake data. At least one support-based recommended action is identified for the at least one support-based evidenced code and a selection of the at least one support-based recommended action is received. Instructions are transmitted to cause execution of the at least one support based recommended action.

### CROSS-REFERENCE TO RELATED APPLICATIONS

This application claims benefit under 35 U.S.C. § 119 (e) to U.S. Provisional Pat. App. Ser. No. 63/549,841, filed Feb. 5, 2024, entitled “Systems and Methods for Automated Code Classification and Natural Language Generation,” the disclosure of which is incorporated herein by reference in its entirety.

### TECHNICAL FIELD

This application relates generally to data intake analysis, and more particularly, to automated data intake analysis for next action identification.

### BACKGROUND

Intake may present a first opportunity for providers to interact with individuals. In a medical context, intake may include a first interaction between a patient and a provider. Decisions made at intake, such as follow-ups scheduled or tests ordered, may establish a roadmap for subsequent interactions. In addition, decisions made during an intake process may define a timeline for a patient to obtain correct and accurate care, with some decisions shortening the timeline and other decisions increasing the timeline.

When ordering or requesting tests, whether diagnostic or otherwise, a provider currently must have a medical necessity justification corresponding to the test. A medical necessity justification may be tied to a suspected diagnosis based on symptoms, complaints, medical history information, etc. that is provided to and/or collected by a provider. To obtain relevant and valid tests, a provider must know what tests are available for ordering, know what tests may be applicable to confirming a suspected diagnosis, and must have medical justification for the suspected diagnosis. A provider may lack knowledge or confidence at each of these steps, resulting in relevant tests that may identify medical issues quicker not being used.

### SUMMARY

In various embodiments, a system including a non-transitory memory and a processor communicatively coupled to the non-transitory memory is disclosed. The processor is configured to read a set of instructions to receive a set of intake data, generate at least one support-based evidenced code based on the set of intake data, identify at least one support-based recommended action for the at least one support-based evidenced code, receive a selection of the at least one support-based recommended action, and transmit instructions to cause execution of the at least one support based recommended action.

In various embodiments, a computer-implemented method is disclosed. The computer-implemented method includes steps of receiving a set of intake data, generating at least one support-based evidenced code based on the set of intake data, identifying at least one support-based recommended action for the at least one support-based evidenced code, receiving a selection of the at least one support-based recommended action, and transmitting instructions to cause execution of the at least one support based recommended action.

In various embodiments, a non-transitory computer-readable medium having instructions stored thereon is disclosed. The instructions, when executed by at least one processor, cause at least one device to perform operations including receiving a set of intake data, generating at least one support-based evidenced code based on the set of intake data, identifying at least one support-based recommended action for the at least one support-based evidenced code, receiving a selection of the at least one support-based recommended action, and transmitting instructions to cause execution of the at least one support based recommended action.

### BRIEF DESCRIPTION OF THE DRAWINGS

The features and advantages of the present invention will be more fully disclosed in, or rendered obvious by the following detailed description of the preferred embodiments, which are to be considered together with the accompanying drawings wherein like numbers refer to like parts and further wherein:

FIG. 1 illustrates a network environment configured to provide next-action recommendation and generate support interfaces, in accordance with some embodiments;

FIG. 2 illustrates a computer system configured to implement one or more processes, in accordance with some embodiments;

FIG. 3 is a flowchart illustrating a data intake and next-action recommendation method, in accordance with some embodiments;

FIG. 4 is a process flow illustrating various steps of the data intake and next-action recommendation method of FIG. 3, in accordance with some embodiments;

FIG. 5 is a flowchart illustrating a support-based code identification process, in accordance with some embodiments;

FIG. 6 is a process flow illustrating various steps of the support-based code identification process, in accordance with some embodiments; and

FIGS. 7A-7C illustrate various interfaces generated during and/or in conjunction with the data intake and next-action recommendation method of FIG. 3, in accordance with some embodiments.

### DETAILED DESCRIPTION

This description of the exemplary embodiments is intended to be read in connection with the accompanying drawings, which are to be considered part of the entire written description. Terms concerning data connections, coupling and the like, such as “connected” and “interconnected,” and/or “in signal communication with” refer to a relationship wherein systems or elements are electrically connected (e.g., wired, wireless, etc.) to one another either directly or indirectly through intervening systems, unless expressly described otherwise. The term “operatively coupled” is such a coupling or connection that allows the pertinent structures to operate as intended by virtue of that relationship.

In the following, various embodiments are described with respect to the claimed systems as well as with respect to the claimed methods. Features, advantages, or alternative embodiments herein may be assigned to the other claimed objects and vice versa. In other words, claims for the systems may be improved with features described or claimed in the context of the methods. In this case, the functional features of the method are embodied by objective units of the systems. While the present disclosure is susceptible to various modifications and alternative forms, specific embodiments are shown by way of example in the drawings and will be described in detail herein. The objectives and advantages of the claimed subject matter will become more apparent from the following detailed description of these exemplary embodiments in connection with the accompanying drawings.

Furthermore, in the following, various embodiments are described with respect to methods and systems for next-action recommendation and support interface generation. In various embodiments, a system is configured to receive intake data. Intake data may include data obtained during one or more initial interactions. For example, in a medical context, intake data may include, but is not limited to, pre-appointment patient-provided information, patient history information, real-time patient-provided information, real-time provider-provided information, test results, etc. The intake data is provided to one or more next-action identification processes configured to generate a set of potential next-actions. In some embodiments, the next-action identification process selects a next-action from a set of available actions. In the medical context, the set of available actions may include test processes available from one or more testing sources (e.g., external testing locations, internal testing capabilities, etc.). The next-action identification process may output one or more selected next actions and corresponding justification data for each of the selected next actions. One or more of the recommended next actions may be selected for execution. In some embodiments, a user interface is generated and updated in real-time as additional intake data is received to adjust the set of recommended next actions and/or corresponding justification data.

In some embodiments, systems and methods for next-action recommendation and support interface generation include generation of one or more interfaces including generated natural language outputs related to at least one of the recommended next actions. For example, one or more trained complex artificial intelligence (AI) systems, for example a complex AI system including a generative AI (GenAI) model, may be utilized to output natural language information related to a recommended next action, justification data corresponding to the recommended next action, and/or other applicable information.

In some embodiments, systems and methods for next-action recommendation and support interface generation include generation of one or more information interfaces. The information interfaces may provide outputs to users (e.g., individuals) based on received intake information. The outputs may include, but are not limited to, analysis of medical necessity for one or more tests, potential diagnosis, and/or any other suitable information. In some embodiments, the information interface includes an indication that outputs are provided for user information purposes only.

In general, a trained function mimics cognitive functions that humans associate with other human minds. In particular, by training based on training data the trained function is able to adapt to new circumstances and to detect and extrapolate patterns.

In general, parameters of a trained function may be adapted by means of training. In particular, a combination of supervised training, semi-supervised training, unsupervised training, reinforcement learning and/or active learning may be used. Furthermore, representation learning (an alternative term is “feature learning”) may be used. In particular, the parameters of the trained functions may be adapted iteratively by several steps of training.

In some embodiments, a trained function may include a neural network, a support vector machine, a decision tree, a Bayesian network, a clustering network, Qlearning, genetic algorithms and/or association rules, and/or any other suitable artificial intelligence architecture. In some embodiments, a neural network may be a deep neural network, a convolutional neural network, a convolutional deep neural network, etc. Furthermore, a neural network may be an adversarial network, a deep adversarial network, a generative adversarial network, etc.

In various embodiments, neural networks which are trained (e.g., configured or adapted) to generate natural language outputs corresponding to recommended next actions, are disclosed. A neural network trained to generate natural language outputs may be referred to as a trained GenAI model. A trained GenAI model may be configured to receive a prompt input and generate a natural language output responsive to the prompt. The prompt input may include one or more definitions, one or more configurations, one or more data points, etc. For example, in some embodiments, a prompt input may include a code selected from a predefined code set.

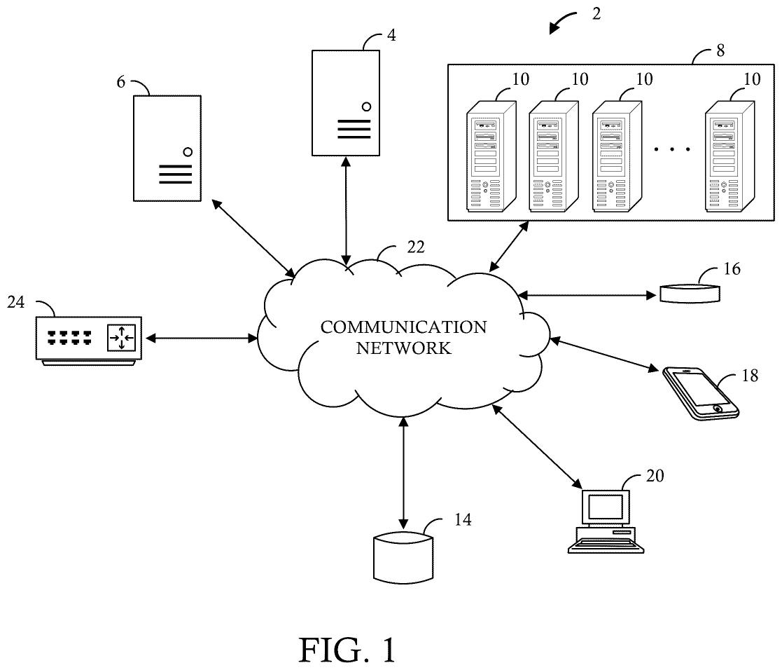

FIG. 1 illustrates a network environment 2 configured to provide supported next action recommendations and generate support interfaces, in accordance with some embodiments. The network environment 2 includes a plurality of devices or systems configured to communicate over one or more network channels, illustrated as a network cloud 22. For example, in various embodiments, the network environment 2 may include, but is not limited to, a supported next action recommendation computing device 4, an application server 6, a cloud-based engine 8 including one or more processing devices 10, a database 14, and/or one or more user computing devices 16, 18, 20 operatively coupled over the network 22. The supported next action recommendation computing device 4, the application server 6, the processing device(s) 10, and/or the user computing devices 16, 18, 20 may each be a suitable computing device that includes any hardware or hardware and software combination for processing and handling information. For example, each computing device may include, but is not limited to, one or more processors, one or more field-programmable gate arrays (FPGAs), one or more application-specific integrated circuits (ASICs), one or more state machines, digital circuitry, and/or any other suitable circuitry. In addition, each computing device may transmit and receive data over the communication network 22.

In some embodiments, each of the supported next action recommendation computing device 4 and the processing device(s) 10 may be a computer, a workstation, a laptop, a server such as a cloud-based server, or any other suitable device. In some embodiments, each of the processing devices 10 is a server that includes one or more processing units, such as one or more graphical processing units (GPUs), one or more central processing units (CPUs), and/or one or more processing cores. Each processing device 10 may, in some embodiments, execute one or more virtual machines. In some embodiments, processing resources (e.g., capabilities) of the one or more processing devices 10 are offered as a cloud-based service (e.g., cloud computing). For example, the cloud-based engine 8 may offer computing and storage resources of the one or more processing devices 10 to the supported next action recommendation computing device 4.

In some embodiments, each of the user computing devices 16, 18, 20 may be a cellular phone, a smart phone, a tablet, a personal assistant device, a voice assistant device, a digital assistant, a laptop, a computer, or any other suitable device. In some embodiments, the application server 6 hosts one or more network environments, such as an electronic health network environment. In some embodiments, the supported next action recommendation computing device 4, the processing devices 10, and/or the application server 6 are operated by the network environment provider, and the user computing devices 16, 18, 20 are operated by users of the network environment. In some embodiments, the processing devices 10 are operated by a third party (e.g., a cloud-computing provider).

Although FIG. 1 illustrates three user computing devices 16, 18, 20, the network environment 2 may include any number of user computing devices 16, 18, 20. Similarly, the network environment 2 may include any number of the supported next action recommendation computing device 4, the application server 6, the processing devices 10, and/or the databases 14. It will further be appreciated that additional systems, servers, storage mechanism, etc. may be included within the network environment 2. In addition, although embodiments are illustrated herein having individual, discrete systems, it will be appreciated that, in some embodiments, one or more systems may be combined into a single logical and/or physical system. For example, in various embodiments, one or more of the supported next action recommendation computing device 4, the application server 6, the database 14, the user computing devices 16, 18, 20, and/or the router 24 may be combined into a single logical and/or physical system. Similarly, although embodiments are illustrated having a single instance of each device or system, it will be appreciated that additional instances of a device may be implemented within the network environment 2. In some embodiments, two or more systems may be operated on shared hardware in which each system operates as a separate, discrete system utilizing the shared hardware, for example, according to one or more virtualization schemes.

The communication network 22 may be a WiFi® network, a cellular network such as a 3GPP® network, a Bluetooth® network, a satellite network, a wireless local area network (LAN), a network utilizing radio-frequency (RF) communication protocols, a Near Field Communication (NFC) network, a wireless Metropolitan Area Network (MAN) connecting multiple wireless LANs, a wide area network (WAN), or any other suitable network. The communication network 22 may provide access to, for example, the Internet.

Each of the user computing devices 16, 18, 20 may communicate with the application server 6 over the communication network 22. For example, each of the user computing devices 16, 18, 20 may be operable to view, access, and interact with a network application, such as a health network application, hosted by the application server 6. The application server 6 may transmit user session data related to a user's activity (e.g., interactions) on the network application.

For example, a user may operate one of the user computing devices 16, 18, 20 to initiate an intake session via a health network application and/or a network accessible interface. The user may, via the network application, perform various operations such as providing intake data, validating intake data, and select recommended next actions for further processing. The network application (e.g., via a user computing device 16, 18, 20) may capture these activities as user session data and transmit the user session data to the supported next action recommendation computing device 4, the application server 6, and/or any other suitable system over the communication network 22.

In some embodiments, the supported next action recommendation computing device 4 may execute one or more models, processes, or algorithms, such as a complex AI system including one or more of a machine learning model, deep learning model, statistical model, a heuristic model, etc., to identify and validate supported next-action recommendations. The supported next action recommendation computing device 4 may transmit one or more recommended next-action identifiers to the application server 6 over the communication network 22, and the application server 6 may display interface elements associated with the recommended next actions, underlying support elements related to the one or more recommended next actions, and/or information associated with the recommended next actions and/or underlying support elements on the network application to the user. For example, the application server 6 may display interface elements associated with support elements corresponding to recommended next actions and/or natural language elements related to recommended next actions or support elements on one or more application interfaces.

The supported next action recommendation computing device 4 is further operable to communicate with the database 14 over the communication network 22. For example, the supported next action recommendation computing device 4 may store data to, and read data from, the database 14. The database 14 may be a remote storage device, such as a cloud-based server, a disk (e.g., a hard disk), a memory device on another application server, a networked computer, or any other suitable remote storage. Although shown remote to the supported next action recommendation computing device 4, in some embodiments, the database 14 may be a local storage device, such as a hard drive, a non-volatile memory, or a USB stick.

In some embodiments, the supported next action recommendation computing device 4 generates configuration data for one or more complex AI systems including, for example, a GenAI model, based on received intake data. The supported next action recommendation computing device 4 and/or one or more of the processing devices 10 may execute one or more complex AI systems based on corresponding configuration data. The supported next action recommendation computing device 4 may store GenAI models and/or configuration data for a complex AI system in a database, such as in the database 14 (e.g., a cloud storage database).

In some embodiments, the supported next action recommendation computing device 4 assigns the complex AI systems (or parts thereof) for execution to one or more processing devices 10. For example, each model of the complex AI system may be assigned to a virtual machine hosted by a processing device 10. The virtual machine may cause the models or parts thereof to execute on one or more processing units such as GPUs. In some embodiments, the virtual machines assign each model (or part thereof) among a plurality of processing units. Based on the output of the models, supported next action recommendation computing device 4 may generate recommended next action data elements including natural language elements corresponding to a recommended next action and/or a support data element.

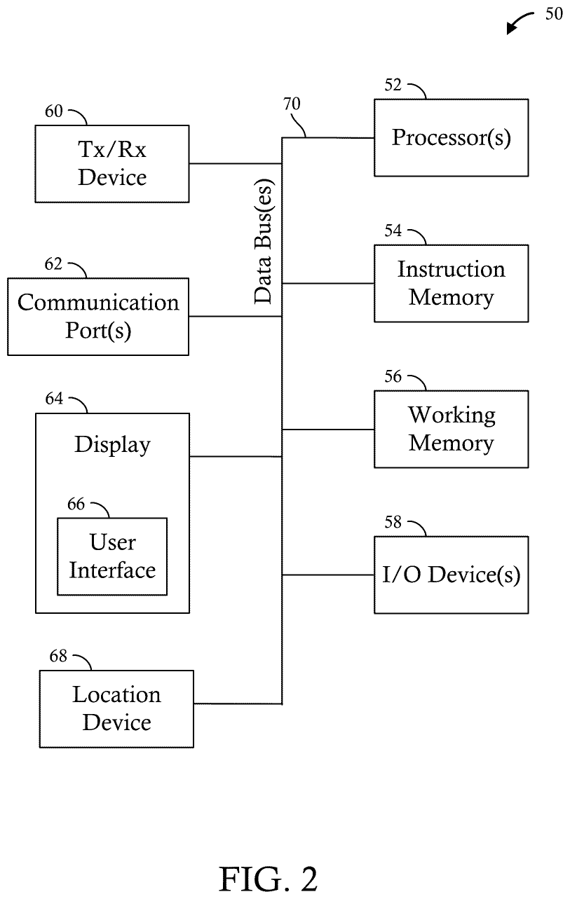

FIG. 2 illustrates a block diagram of a computing device 50, in accordance with some embodiments. In some embodiments, each of the supported next action recommendation computing device 4, the application server 6, the one or more processing devices 10, and/or the user computing devices 16, 18, 20 in FIG. 1 may include the features shown in FIG. 2. Although FIG. 2 is described with respect to certain components shown therein, it will be appreciated that the elements of the computing device 50 may be combined, omitted, and/or replicated. In addition, it will be appreciated that additional elements other than those illustrated in FIG. 2 may be added to the computing device.

As shown in FIG. 2, the computing device 50 may include one or more processors 52, an instruction memory 54, a working memory 56, one or more input/output devices 58, a transceiver 60, one or more communication ports 62, a display 64 with a user interface 66, and an optional location device 68, all operatively coupled to one or more data buses 70. The data buses 70 allow for communication among the various components. The data buses 70 may include wired, or wireless, communication channels.

The one or more processors 52 may include any processing circuitry operable to control operations of the computing device 50. In some embodiments, the one or more processors 52 include one or more distinct processors, each having one or more cores (e.g., processing circuits). Each of the distinct processors may have the same or different structure. The one or more processors 52 may include one or more central processing units (CPUs), one or more graphics processing units (GPUs), application specific integrated circuits (ASICs), digital signal processors (DSPs), a chip multiprocessor (CMP), a network processor, an input/output (I/O) processor, a media access control (MAC) processor, a radio baseband processor, a co-processor, a microprocessor such as a complex instruction set computer (CISC) microprocessor, a reduced instruction set computing (RISC) microprocessor, and/or a very long instruction word (VLIW) microprocessor, or other processing device. The one or more processors 52 may also be implemented by a controller, a microcontroller, an application specific integrated circuit (ASIC), a field programmable gate array (FPGA), a programmable logic device (PLD), etc.

In some embodiments, the one or more processors 52 are configured to implement an operating system (OS) and/or various applications. Examples of an OS include, for example, operating systems generally known under various trade names such as Apple macOS™, Microsoft Windows™, Android™, Linux™, and/or any other proprietary or open-source OS. Examples of applications include, for example, network applications, local applications, data input/output applications, user interaction applications, etc.

The instruction memory 54 may store instructions that are accessed (e.g., read) and executed by at least one of the one or more processors 52. For example, the instruction memory 54 may be a non-transitory, computer-readable storage medium such as a read-only memory (ROM), an electrically erasable programmable read-only memory (EEPROM), flash memory (e.g. NOR and/or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, a removable disk, CD-ROM, any non-volatile memory, or any other suitable memory. The one or more processors 52 may be configured to perform a certain function or operation by executing code, stored on the instruction memory 54, embodying the function or operation. For example, the one or more processors 52 may be configured to execute code stored in the instruction memory 54 to perform one or more of any function, method, or operation disclosed herein.

Additionally, the one or more processors 52 may store data to, and read data from, the working memory 56. For example, the one or more processors 52 may store a working set of instructions to the working memory 56, such as instructions loaded from the instruction memory 54. The one or more processors 52 may also use the working memory 56 to store dynamic data created during one or more operations. The working memory 56 may include, for example, random access memory (RAM) such as a static random access memory (SRAM) or dynamic random access memory (DRAM), Double-Data-Rate DRAM (DDR-RAM), synchronous DRAM (SDRAM), an EEPROM, flash memory (e.g. NOR and/or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, a removable disk, CD-ROM, any non-volatile memory, or any other suitable memory. Although embodiments are illustrated herein including separate instruction memory 54 and working memory 56, it will be appreciated that the computing device 50 may include a single memory unit configured to operate as both instruction memory and working memory. Further, although embodiments are discussed herein including non-volatile memory, it will be appreciated that computing device 50 may include volatile memory components in addition to at least one non-volatile memory component.

In some embodiments, the instruction memory 54 and/or the working memory 56 includes an instruction set, in the form of a file for executing various methods, such as methods for supported next-action recommendation and support interface generation, as described herein. The instruction set may be stored in any acceptable form of machine-readable instructions, including source code or various appropriate programming languages. Some examples of programming languages that may be used to store the instruction set include, but are not limited to: Java, JavaScript, C, C++, C#, Python, Objective-C, Visual Basic, .NET, HTML, CSS, SQL, NoSQL, Rust, Perl, etc. In some embodiments a compiler or interpreter is configured to convert the instruction set into machine executable code for execution by the one or more processors 52.

The input-output devices 58 may include any suitable device that allows for data input or output. For example, the input-output devices 58 may include one or more of a keyboard, a touchpad, a mouse, a stylus, a touchscreen, a physical button, a speaker, a microphone, a keypad, a click wheel, a motion sensor, a camera, and/or any other suitable input or output device.

The transceiver 60 and/or the communication port(s) 62 allow for communication with a network, such as the communication network 22 of FIG. 1. For example, if the communication network 22 of FIG. 1 is a cellular network, the transceiver 60 is configured to allow communications with the cellular network. In some embodiments, the transceiver 60 is selected based on the type of the communication network 22 the computing device 50 will be operating in. The one or more processors 52 are operable to receive data from, or send data to, a network, such as the communication network 22 of FIG. 1, via the transceiver 60.

The communication port(s) 62 may include any suitable hardware, software, and/or combination of hardware and software that is capable of coupling the computing device 50 to one or more networks and/or additional devices. The communication port(s) 62 may be arranged to operate with any suitable technique for controlling information signals using a desired set of communications protocols, services, or operating procedures. The communication port(s) 62 may include the appropriate physical connectors to connect with a corresponding communications medium, whether wired or wireless, for example, a serial port such as a universal asynchronous receiver/transmitter (UART) connection, a Universal Serial Bus (USB) connection, or any other suitable communication port or connection. In some embodiments, the communication port(s) 62 allows for the programming of executable instructions in the instruction memory 54. In some embodiments, the communication port(s) 62 allow for the transfer (e.g., uploading or downloading) of data, such as machine learning model training data.

In some embodiments, the communication port(s) 62 are configured to couple the computing device 50 to a network. The network may include local area networks (LAN) as well as wide area networks (WAN) including without limitation Internet, wired channels, wireless channels, communication devices including telephones, computers, wire, radio, optical and/or other electromagnetic channels, and combinations thereof, including other devices and/or components capable of/associated with communicating data. For example, the communication environments may include in-body communications, various devices, and various modes of communications such as wireless communications, wired communications, and combinations of the same.

In some embodiments, the transceiver 60 and/or the communication port(s) 62 are configured to utilize one or more communication protocols. Examples of wired protocols may include, but are not limited to, Universal Serial Bus (USB) communication, RS-232, RS-422, RS-423, RS-485 serial protocols, Fire Wire, Ethernet, Fibre Channel, MIDI, ATA, Serial ATA, PCI Express, T-1 (and variants), Industry Standard Architecture (ISA) parallel communication, Small Computer System Interface (SCSI) communication, or Peripheral Component Interconnect (PCI) communication, etc. Examples of wireless protocols may include, but are not limited to, the Institute of Electrical and Electronics Engineers (IEEE) 802.xx series of protocols, such as IEEE 802.11a/b/g/n/ac/ag/ax/be, IEEE 802.16, IEEE 802.20, GSM cellular radiotelephone system protocols with GPRS, CDMA cellular radiotelephone communication systems with 1×RTT, EDGE systems, EV-DO systems, EV-DV systems, HSDPA systems, Wi-Fi Legacy, Wi-Fi 1/2/3/4/5/6/6E, wireless personal area network (PAN) protocols, Bluetooth Specification versions 5.0, 6, 7, legacy Bluetooth protocols, passive or active radio-frequency identification (RFID) protocols, Ultra-Wide Band (UWB), Digital Office (DO), Digital Home, Trusted Platform Module (TPM), ZigBee, etc.

The display 64 may be any suitable display, and may display the user interface 66. The user interfaces 66 may enable user interaction with recommended next actions and/or support elements. For example, the user interface 66 may be a user interface for an application of a network environment operator that allows a user to view and interact with the operator's website. In some embodiments, a user may interact with the user interface 66 by engaging the input-output devices 58. In some embodiments, the display 64 may be a touchscreen, where the user interface 66 is displayed on the touchscreen.

The display 64 may include a screen such as, for example, a Liquid Crystal Display (LCD) screen, a light-emitting diode (LED) screen, an organic LED (OLED) screen, a movable display, a projection, etc. In some embodiments, the display 64 may include a coder/decoder, also known as Codecs, to convert digital media data into analog signals. For example, the visual peripheral output device may include video Codecs, audio Codecs, or any other suitable type of Codec.

The optional location device 68 may be communicatively coupled to a location network and operable to receive position data from the location network. For example, in some embodiments, the location device 68 includes a GPS device configured to receive position data identifying a latitude and longitude from one or more satellites of a GPS constellation. As another example, in some embodiments, the location device 68 is a cellular device configured to receive location data from one or more localized cellular towers. Based on the position data, the computing device 50 may determine a local geographical area (e.g., town, city, state, etc.) of its position.

In some embodiments, the computing device 50 is configured to implement one or more modules or engines, each of which is constructed, programmed, configured, or otherwise adapted, to autonomously carry out a function or set of functions. A module/engine may include a component or arrangement of components implemented using hardware, such as by an application specific integrated circuit (ASIC) or field-programmable gate array (FPGA), for example, or as a combination of hardware and software, such as by a microprocessor system and a set of program instructions that adapt the module/engine to implement the particular functionality, which (while being executed) transform the microprocessor system into a special-purpose device. A module/engine may also be implemented as a combination of the two, with certain functions facilitated by hardware alone, and other functions facilitated by a combination of hardware and software. In certain implementations, at least a portion, and in some cases, all, of a module/engine may be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each module/engine may be realized in a variety of physically realizable configurations, and should generally not be limited to any particular implementation exemplified herein, unless such limitations are expressly called out. In addition, a module/engine may itself be composed of more than one sub-modules or sub-engines, each of which may be regarded as a module/engine in its own right. Moreover, in the embodiments described herein, each of the various modules/engines corresponds to a defined autonomous functionality; however, it should be understood that in other contemplated embodiments, each functionality may be distributed to more than one module/engine. Likewise, in other contemplated embodiments, multiple defined functionalities may be implemented by a single module/engine that performs those multiple functions, possibly alongside other functions, or distributed differently among a set of modules/engines than specifically illustrated in the embodiments herein.

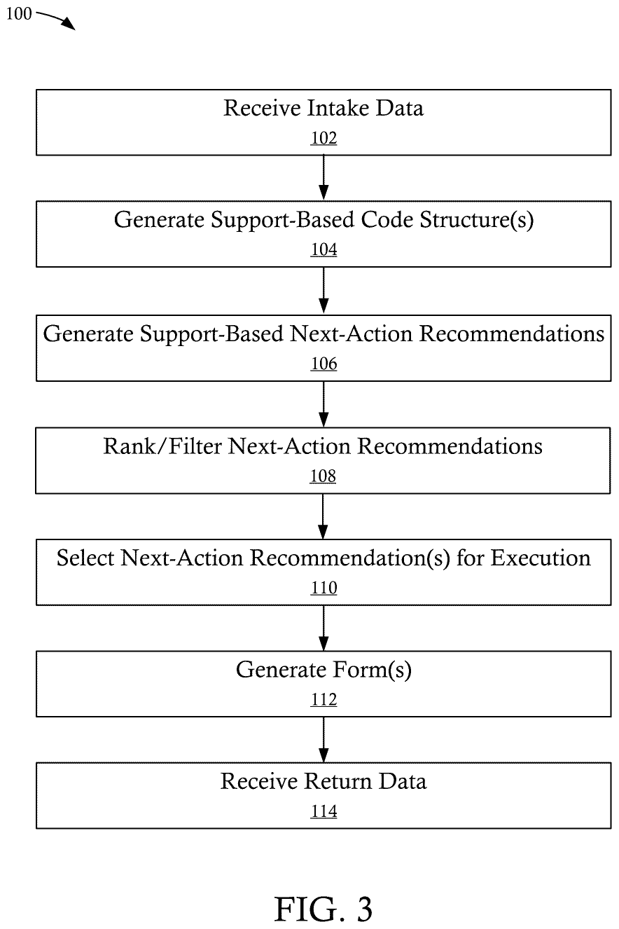

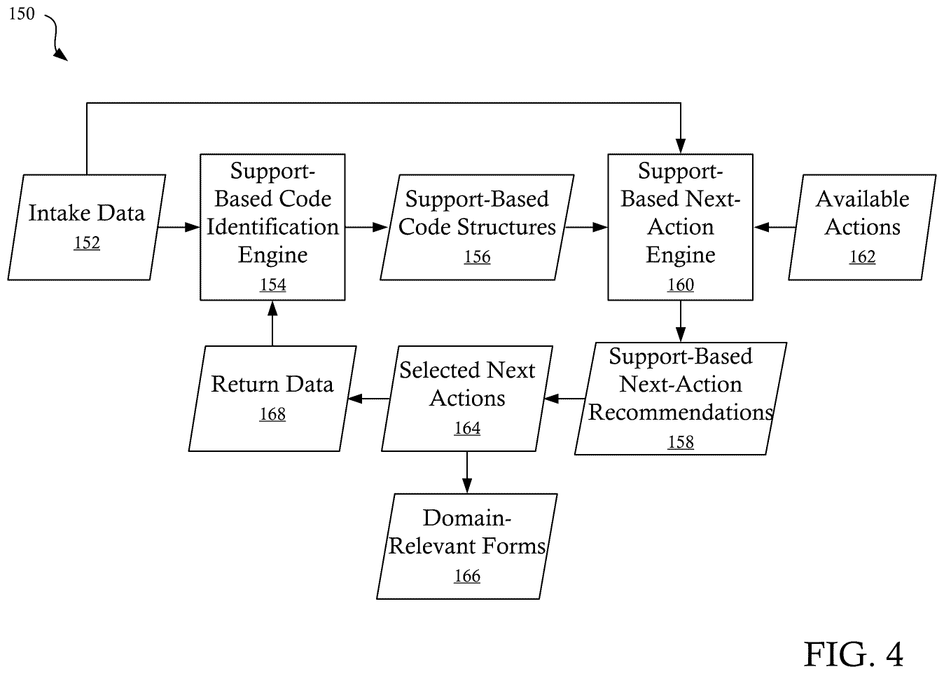

FIG. 3 is a flowchart illustrating a support interface generation method 100, in accordance with some embodiments. FIG. 4 is a process flow 150 illustrating various steps of the support interface generation method 100, in accordance with some embodiments. The support interface generation method 100 may be executed by any suitable system, device, module, engine, etc., and/or combination thereof. For example, in some embodiments, the support interface generation method 100 may be executed by one or more of the supported next action recommendation computing device 4, the application server 6, the one or more processing devices 10, and/or any other suitable systems.

At step 102, intake data 152 is received. The intake data 152 may include any suitable data related to an initial interaction. An initial interaction may include a first interaction between two entities, a first interaction in a series of interactions related to one or more subjects, and/or a first interaction in subsequent series of interactions. For example, in a medical context, an initial interaction may include, but is not limited to: a first interaction between a patient and a provider, such as a new patient interaction; an interaction including a first complaint of a symptom, suspected medical issue, etc.; an interaction including a first disclosure of additional symptoms or concerns related to a prior complaint; etc.

In some embodiments, intake data 152 includes one or more data elements related to a specific knowledge domain, such as a medical knowledge domain. For example, the intake data 152 may be collected prior to and/or during an interaction between a patient and a medical provider and may be related to one or more medical complaints or potentialities. In the context of a medical knowledge domain, the intake data 152 may include data elements such as patient demographic information, medical history information, current medical status, patient complaints prior diagnosis, observations, testing results, etc.

Intake data 152 may be categorized and/or segregated into two or more categories, such as, for example, protected information and non-protected information. As one non-limiting example, protected information may include information covered by one or more data protection schemes, such as information protection established under HIPPA (the Health Insurance Portability and Accountability Act). Intake data 152 may be generated by an automated process (e.g., obtained from one or more electronic storage systems), generated by a computer-implemented process (e.g., obtained via entry into one or more electronic forms), and/or otherwise entered into a computing system as one or more data elements.

In some embodiments, intake data 152 may be generated based on an iterative (e.g., responsive) intake process configured to identify additional expected, required, and/or desired intake data elements based on currently received intake data 152. An iterative intake process may be configured to identify missing data elements from one or more data sources (e.g., missing electronic intake forms, missing entries within electronic intake forms, missing data elements within a defined data structure, etc.) and/or may be configured to request additional data elements based on potential classifications of intake data 152. As one non-limiting example, in a medical domain context, intake data 152 may include data elements that suggest one or more potential diagnoses (as discussed in greater detail below with respect to step 104), but that require additional information to confirm or eliminate. An iterative intake data process may be configured to identify data gaps where additional information may be useful in confirming or rejecting certain next-actions and may prompt a user (e.g., patient, provider, etc.) to collect additional intake data 152.

In some embodiments, intake data 152 may be obtained through an iterative intake process. For example, one or more first intake interfaces may be generated to collect a first set of intake data. The first set of intake data may be analyzed to identify additional intake data necessary and/or desirable for use in additional downstream processes, such as, for example, code identification as discussed below in conjunction with step 104. One or more second intake interfaces may be generated to facilitate collection of one or more additional sets of intake data that may supplement and/or replace data elements in the first set of intake data to provide a more robust analysis at subsequent steps.

In some embodiments, intake data 152 may include data received from one or more data stores, such as, for example, one or more databases 14. For example, intake data 152 may include historical data (e.g., patient history, prior testing, prior diagnoses, etc.) generated prior to and/or simultaneously with the intake interaction represented, at least in part, by the intake data 152.

At step 104, one or more support-based identified codes 156 are generated. The support-based identified codes 156 include elements representative of recommended next actions for a corresponding knowledge domain identified based on the intake data 152. The identified codes are support-based as they rely on and provide domain-specific data elements that suggest or support each identified code. The domain-specific requirements may include one or more category of requirements, such as, for example, one or more justification categories.

As one non-limiting example, in some embodiments, the intake data 152 and the support-based identified codes are related to a medical domain (e.g., a medical code set). Prior to and/or during an intake interaction, intake data 152 may be obtained. Intake data 152 may include data points or elements that, when considered collectively, suggest or relate to one or more potential diagnoses represented within and/or by the medical code set. For example, in a medical context, a combination of intake data 152 identifying symptoms or complaints including feelings of thirst, frequent urination, loss of weight, and mood changes may indicate one or more potential diagnoses, such as type 1 diabetes, type 2 diabetes, anemia, etc. A set of support-based identified codes 156 may be generated identifying potential code classifications (e.g., diagnoses) based on the corresponding intake data (e.g., support elements). The support-based identified codes 156 are generated based on the provided intake data 152 (e.g., the set of symptoms and complaints) and corresponding intake data elements (e.g., specific symptoms) are associated with each identified code.

In some embodiments, the support-based identified codes 156 are generated by a support-based code identification engine 154 configured to receive the intake data 152 and output the set of support-based identified codes 156. The support-based code identification engine 154 may include one or more of a rules-based engine, a heuristic-based engine, a model-based engine, a machine learning-based engine, etc. As one non-limiting example, in some embodiments, the support-based code identification engine 154 includes a rules-based module. As another non-limiting example, in some embodiments, the support-based code identification engine 154 includes a trained multi-classification model configured to classify intake data into at least one of a plurality of potential classifications.

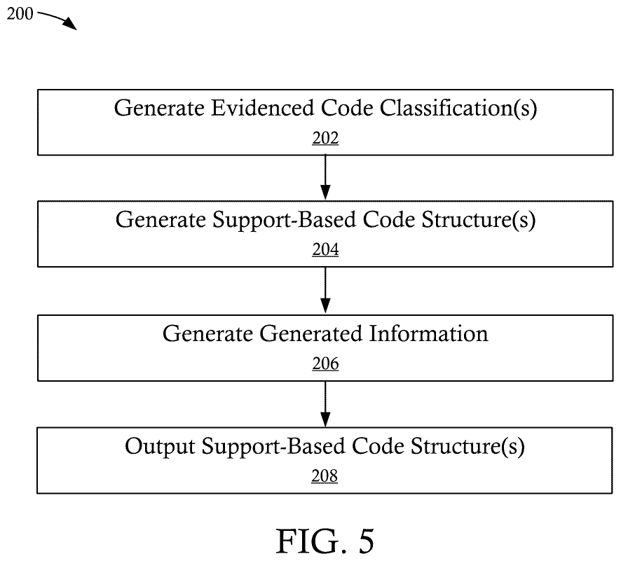

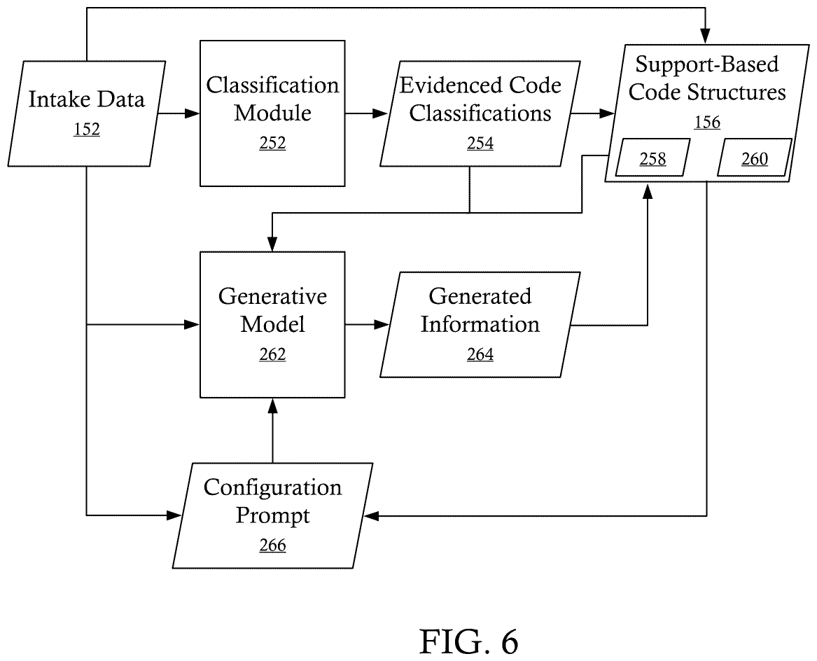

FIG. 5 is a flowchart illustrating a support-based code identification process 200, in accordance with some embodiments. FIG. 6 is a process flow 250 illustrating various steps of the support-based code identification process 200, in accordance with some embodiments. The support-based code identification process 200 may be implemented by any suitable system, module, engine, etc., such as the support-based code identification engine 154. At step 202, a set of evidenced code classifications 254 is generated. The set of evidenced code classifications 254 include one or more codes selected from a predetermined code set that potentially apply to the intake data 152 based on one or more data elements included within the intake data 152. For example, in a medical domain, the predetermined code set may include one or more medical code sets (e.g., ICD-10 codes), a specific portion of a medical code set (e.g., only diagnostic codes within ICD-10), etc. The identified codes correspond to one or more intake data elements, e.g., one or more symptoms, that support or suggest the corresponding predetermined code.

In some embodiments, set of evidenced code classifications 254 include codes that correspond to a predetermined minimal number of intake data elements. For example, a single data element may not be sufficient to confidently identify any code in a predetermined code set, as the single data element may potentially correspond to multiple codes. A predetermined minimal number of matching intake data elements may be required to include a code in the set of potential code classifications. The predetermined minimal number may static across a predetermined code set (e.g., each code in a predetermined code set requires N matching data elements to be included in the set of evidenced code classifications 254) and/or may be code-specific (e.g., each code in the predetermined code set defines the number of matching data elements required for that code (or set of codes) to be included in the set of evidenced code classifications 254). It will be appreciated that the predetermined minimal number of intake data elements (e.g., the confidence threshold) may be defined by the predetermined code set, defined by one or more iterative/training processes, and/or otherwise selected for application to one or more predetermined code sets and/or codes.

In some embodiments, the set of evidenced code classifications 254 is based on multiple dimensions represented within intake data 152. For example, the set of evidenced code classifications 254 may be generated based on symptom matching, symptom time frames, prior health data, interaction-obtained data points (e.g., in-office blood pressure, pulse, etc.), and/or other suitable dimensions relevant to a given domain, such as a medical domain. In some embodiments, the set of evidenced code classifications 254 is generated based on a spectrum analysis configured extract, generate, and/or apply multiple dimensions of consideration from the intake data 152. For example, in the context of a medical domain, the set of evidenced code classifications 254 may be generated based on determinations within a spectrum having multiple dimensions defined by multiple potential data points included within the intake data 152.

In some embodiments, the set of evidenced code classifications 254 is identified by a classification module 252. The classification module 252 may be configured to receive the intake data 152 and output the set of evidenced code classifications 254. For example, a set of predetermined heuristic rules may be applied to generate one or more classifications (e.g., one or more classification scores) corresponding to each code in the predetermined code set. As another example, a set of trained models and/or validated models may be applied to generate one or more classifications. In some embodiments, the classification module 252 may be configured to generate a confidence score for each code in a set of predetermined codes. Codes having a confidence score above a predetermined threshold may be selected for inclusion in the set of evidenced code classifications 254. Confidence scores may be defined for an entire predetermined code set, portions of the predetermined code set, and/or for individual codes within the predetermined code set. The confidence scores may be generated based on a comparison to historical data in the corresponding domain, an iterative training process, and/or other threshold-setting considerations.

In some embodiments, classification determinations applied by a classification module 252 are generated utilizing one or more verified information sources. For example, in the context of a medical domain, one or more heuristic rules and/or trained models may be generated based on verified, medical information sources. Verified information sources may include domain- specific, trusted sources that provide verifiable, domain-specific information that may be applied to identify one or more codes within a subset of codes based on the intake data 152.

In the context of a medical domain, the set of evidenced code classifications 254 may include diagnostic codes selected from a corresponding diagnostic code set, such as ICD-10, Health Care Common Procedure Coding System (HCPCS), Current Procedure Terminology (CPT), Code on Dental Procedures and Nomenclature (CDT), National Drug Codes (NDC), SNOMED, etc. A confidence score may be selected for a code set and/or for each code within the code set and/or a set of heuristic rules may be applied to identify a code within a corresponding code set. The selected codes included in the set of evidenced code classifications 254 may represent potential diagnoses for a patient corresponding to the intake data 152.

In some embodiments, a set of evidenced code classifications 254 may be generated and/or modified by the intake data 152, such as current symptoms, in view of historical data, such as historical symptoms, disease states, etc. For example, a set of evidenced code classifications 254 generated for intake data 152 include a first set of symptoms each having a similar, recent temporal dimension (e.g., symptoms occurring together and within a recent time frame such as the past X days) may generate a first set of evidenced code classifications 254 while intake data 152 including the same first set of symptoms but each having different temporal periods (e.g., symptoms occurring at progressive or disparate time periods) may generate a second set of evidenced code classifications 254.

At step 204, one or more support-based code structures 156 may be generated. The support-based code structures 156 may include a data structure including an evidenced code 258 from the set of evidenced code classifications 254, underlying support elements 260 extracted from and/or identified in the intake data 152, and/or additional data (such as generated information 264 generated for the corresponding code classification, as discussed in greater detail below). For example, in embodiments including a medical domain code set, such as the ICD-10 code set, a support-based code structures 156 may include one or more domain-specific evidenced codes 258 (e.g., one or more ICD-10 codes) identified at step 204. A support-based identified code structure 156 may be limited to only a single corresponding code selected from the set of evidenced code classifications 254 and/or may include multiple codes that each correspond to the underlying support elements 260 and/or are interrelated.

In some embodiments, a domain-specific required level of support is provided for the evidenced code 258 in each support-based identified code structures 156. For example, in the context of a medical domain, support elements 260 extracted from the intake data 152 that informed the selection of and/or are related to the evidenced code 258 provide medical necessity support for selection of the evidenced code 258. Although specific embodiments are discussed herein, it will be appreciated that the domain-specific required level of support may be any predetermined level of support required within the domain and expressed by the classification module 252.

At step 206, generated information 264 is generated for each support-based code structure 156. For example, generated information may be related to an evidenced code 258, one or more underlying support elements 260, and/or any other suitable portion of the support-based code structure 156. The generated information 264 may be generated using any suitable process, such as, for example, a generative language process. In some embodiments, the generated information 264 includes natural language (e.g., text or speech) information generated by one or more generative language models. The generative language model may include any suitable generative language model, such as, for example, a GPT-based model (e.g., ChatGPT-3, ChatGPT-3.5, ChatGPT-4), a Glo Ve-based model, a LLaMA-based model, etc.

In some embodiments, a prompt 266 is generated from one or more elements of the intake data 152 and/or one or more portions of the support-based code structure 156. A prompt 266 may be provided to a generative model 262. The prompt 266 may include one or more configuration elements (e.g., one or more elements, such as textual elements, to configure the generative language model), one or more examples, one or more definitions, one or more target elements (e.g., a recommended action, a code, etc.). The prompt 266 may be provided to a generative language model, which is configured based on the configuration and/or definition elements in the prompt 266 and generates natural language content corresponding to the one or more target elements.

In some embodiments, the prompt 266 is configured to focus or limit content generated by the generative language model to a predetermined domain, such as a domain corresponding to the selected code set. For example, in the context of a medical domain, a selected code set may include a medical diagnostic code set (e.g., ICD-10) and the prompt 266 may be configured to limit generated content to the medical domain, a medical diagnostic domain, etc. In some embodiments, the inclusion of an evidenced code from a domain-specific code set provides for precise calibration of a corresponding generative model, such as a generative language model. For example, some ICD-10 codes correspond to precise disease or diagnostic states (e.g., differentiating different versions of similar diagnoses) and inclusion of an ICD-10 code and/or a textual description of the corresponding ICD-10 code (e.g., the medically-precise disease state description used in the ICD-10 code set) in a prompt 266 provides for targeted configuration of the generative language model to generate information for the corresponding precise diagnostic state. The inclusion of a domain-specific code further facilitates configuration of a generative model through one or more prompts to generate evidenced, accurate, and reliable information related to the corresponding diagnostic state (e.g., reducing or eliminating the potentiality for hallucinations or other undesirable generated data).

In some embodiments, the prompt 266 includes one or more configuration elements to configure the generative language model in a predetermined, domain-specific persona. For example, in the context of a medical domain, the prompt 266 may include one or more configuration elements to configure the generative language model to provide natural language content from a selected persona (e.g., in the view of) within the medical domain, such as a sub-specialist (e.g., cardiologist, neurologist, etc.).

In some embodiments, the generated information 264 includes domain-specific content elements. For example, in the context of a medical domain, the generated information 264 may include, but is not limited to, potential diagnosis, potential treatment protocols for each of the potential diagnosis, medications commonly prescribed for the potential diagnosis, lifestyle changes commonly suggested in conjunction with the potential diagnosis, sample conversations between providers (e.g., general providers, specialty providers) regarding each potential diagnosis, etc. The generated information 264 may be based on intake data 152 and/or one or more evidenced codes 258. As one non-limiting example, one or more prompts 266 may be generated to configure a generative model to output predicted diagnosis based on selected evidenced codes 258 and generated natural language information related to each of the predicted diagnosis.

In some embodiments, the generated information 264 includes recommended specialty referrals. For example, in the context of a medical domain, the generated content may identify specialist (e.g., cardiologist, endocrinologist, neurologist, etc.) that are associated with evidenced codes 258. The specialty referrals may allow a provider to identify the type of specialist required to perform additional testing and/or additional examinations based on the evidenced codes 258 and the corresponding predicted diagnoses. In some embodiments, the specialty referrals may be associated with other generated content, such as domain-specific content from a first perspective or persona.

At step 208, one or more generated support-based code structures 156 are output for use in additional downstream processing, as discussed in greater detail below. Each of the generated support-based code structures 156 may include an associated evidenced code 258 selected from a domain-specific code set, a set of underlying support elements 260 extracted from the intake data 152 that evidence and/or support the evidenced code 258, and/or generated information such as natural language information representative of and/or related to the evidenced code 258 and/or the underlying support elements 260.

With reference against to FIGS. 3-4, at step 106, one or more support-based next-action recommendations 158 are generated. The support-based next-action recommendations 158 include recommended next actions within a corresponding knowledge domain identified for execution based on the intake data 152 and/or the support-based code structures 156. The recommended next-actions 158 are support-based as they rely on and provide domain-specific requirements for each recommended action. The domain-specific requirements may include one or more category of requirements, such as, for example, one or more justifications.

As one non-limiting example, in some embodiments, the intake data 152, the support-based code structures 156, and the support-based next-action recommendations 158 are each related to a medical domain. Prior to and/or during an intake interaction, intake data 152 may be obtained. Intake data 152 may include data points or elements that, when considered collectively, suggest or relate to one or more evidenced codes 258. A set of support-based next-action recommendations 158 may be generated by selecting available actions (e.g., lab tests) to be executed to confirm or reject evidenced codes 258. The support-based next-action recommendations 158 are generated based on the provided intake data 152 (e.g., the set of symptoms and complaints) and/or elements of a corresponding support-based code structure 156.

In some embodiments, the support-based next-action recommendations 158 are generated by a support-based next-action engine 160 configured to receive the intake data 152 and/or the support-based code structures 156 and output the set of support-based next-action recommendations 158. The support-based next-action engine 160 may include one or more of a rules-based engine, a heuristic-based engine, a model-based engine, a machine learning-based engine, etc. As one non-limiting example, in some embodiments, the support-based next-action engine 160 includes a rules-based module. As another non-limiting example, in some embodiments, the support-based next-action engine 160 includes a trained multi-classification model configured to classify intake data into at least one of a plurality of potential classifications.

In some embodiments, the set of support-based next-action recommendations 158 is selected from a set of available actions 162. The set of available actions 162 may include data elements representative of one or more actions that may be taken responsive to selection of one or more codes in the predetermined code set. The set of available actions 162 may include one or more executable activities or operations configured to obtain additional data elements (e.g., return data elements) to confirm and/or reject one or more of the evidenced codes 258. For example, in the context of a medical domain, the set of available actions 162 may include one or more tests, such as blood tests, diagnostic tests, etc. that may be implemented to collect additional medical data for determining the accuracy (e.g., confirming) one or more potential diagnoses represented by one or more evidenced codes 258.

In some embodiments, the set of available actions 162 may include one or more categories of available actions. For example, in the context of a medical domain, the set of available actions 162 may include, but is not limited to, on-site testing actions, off-site testing actions, on-site diagnostic actions, off-set diagnostic actions, etc. In some embodiments, the set of available actions 162 may be tied to one or more entities configured to administer, facilitate, and/or otherwise provide one or more available actions. For example, in the context of a medical domain, the set of available actions 162 may be classified by device (e.g., a corresponding testing device or “stack” configured to execute one or more tests sequentially and/or simultaneously), testing provider (e.g., a corresponding testing location or provider that is capable of administering one or more diagnostic tests or procedures such as a lab, specialty clinic, etc.), provider (e.g., a corresponding provider type), etc.

One or more support-based next-action recommendations 158 may be identified based on the set of support-based code structures 156 and the set of available actions 162. The set of support-based next-action recommendations 158 may include actions in the set of available actions 162 that correspond to at least evidenced code 258. For example, in the context of a medical domain, a support-based next-action recommendation 158 may be generated by identifying one or more actions, such as one or more tests, that collect and/or generate data useful or tied to confirming and/or rejecting a potential diagnosis represented by a corresponding evidenced code 258.

In some embodiments, the set of support-based next-action recommendations 158 may include actions configured to obtain patient data that includes and/or is related to one or more diagnostic measures used to verify and/or reject a given diagnosis, e.g., a corresponding evidenced code 258. For example, the support-based next-action recommendations 158 may include actions that generate testing data relevant to each diagnosis embodied in the identified codes selected based on the intake data 152.

At optional step 108, each of the support-based next-action recommendations 158 may be ranked and/or filtered based on one or more criteria. For example, support-based next-action recommendations 158 may have one or more metrics associated therewith, such as an efficacy value, a return on investment value, a time to execute, etc. A set of generated support-based next-action recommendations 158 may be filtered to remove support-based next-action recommendations 158 having one or more metrics below or above a predetermined threshold value and/or may be ranked according to one or more metrics. For example, in some embodiments, a set of generated support-based next-action recommendations 158 may be ranked according to a return on investment value indicating a total value for executing a test (e.g., insurance payment value less a cost to operate a testing device and/or order a test). Although specific embodiments are discussed herein, it will be appreciated that any suitable ranking metrics may be utilized.

In some embodiments, support-based next-action recommendations 158 may grouped prior to and/or as part of a ranking process, such as grouping by device capable of performing the corresponding actions. For example, in the medical domain, testing devices (e.g., testing stacks) may be configured to run multiple tests, either simultaneously and/or selectively. When multiple support-based next-action recommendations 158 may be executed on a single testing device simultaneously, the corresponding support-based next-action recommendations 158 may be ranked collectively, e.g., the metrics for each action that can be simultaneously executed on a single testing device may be aggregated and the group of corresponding support-based next-action recommendations 158 may be ranked based on the corresponding, aggregated testing device rank. In some embodiments, a single support-based next-action recommendations 158 may be aggregated into multiple testing devices, with each testing device grouping being independently ranked.

At optional step 110, one or more of the recommended next-action recommendations 158 are selected for execution. The selected next actions 164 may be executed by one or more automated, hybrid, and/or manual processes. For example, in embodiments including selected next actions 164 including testing actions (e.g., lab testing, outpatient testing, etc.), each of the testing actions may be executed, for example, by an internal process (e.g., a process associated with the provider included in the current interaction), an external process (e.g., a process associated with a provider other than the provider of the current interaction), a self-administered process, etc.

In some embodiments, an automated order is generated for each action in the selected next actions 164. Automated orders may include necessary documentation for executing a test, such as a referral and/or medical order. The automated orders may be transmitted to systems associated with internal and/or external testing processes. As one non-limiting example, when a selected next action 164 includes a test to be performed by an external testing provider, an electronic order for the corresponding test is generated and transmitted to a system associated with the external testing provider. The external testing provider may implement additional processes to execute the test (e.g., scheduling processes, test collection processes, data processing processes, etc.) based on the automated order.

At optional step 112, one or more domain-relevant forms 166 are automatically generated including data associated with one or more of the selected next actions 164. For example, in some embodiments, one or more billing forms for billing one or more payment entities may be generated by populating a form template with data associated with one or more of the selected next actions 164. The domain-relevant forms 166 may include one or more data elements evidencing and/or related to the domain-specific required level of support for the selected next actions 164. For example, in the context of a medical domain, a domain-relevant form 166 may include a billing form including data elements evidencing and/or supporting the medical necessity determination for each of the selected next actions 164.

At optional step 114, return data 168 for one or more selected next actions 164 is received. The return data 168 may include, for example, test results generated by one or more tests executed in accordance with step 110. The return data 168 may be provided for each selected next action 164 and/or for a subset of the selected next actions 164. In the context of a medical domain, the return data 168 may include, but is not limited to, lab results (e.g., blood tests, urine tests, etc.), automatic nervous system (ANS) testing results, diagnostic testing results, observation results, etc. Return data 168 may be provided in any suitable format and may be processed in the received format and/or converted into a second format suitable for use by one or more systems and/or processes.

At optional step 116, the each of the evidenced codes 258 is verified, rejected, and/or revised based on the intake data 152 and the return data 168. For example, the intake data 152 and the return data 168 may be iteratively provided to the support-based code identification engine 154 to iteratively identify evidenced codes 258. Each of the evidenced codes 258 may be verified, rejected, and/or revised based on additional data. For example, received lab testing results may exclude a potential diagnosis (and a corresponding evidenced code 258), may further evidence (e.g., verify) a previously evidenced code, and/or may result in a revised code (e.g., related, but different code) being selected. In some embodiments, the iterative application of additional return data 168 provides for calibration of generated content from the generative models.

In some embodiments, one or more revised codes including one or more evaluation and management codes are generated. Evaluation and management codes may include codes, such as ICD-10 codes, corresponding to higher level monitoring and/or operations with respect to a corresponding diagnosis. For example, with respect to ICD-10 codes, evaluation and management codes may provide for additional evaluation and/or data gathering for a patient with respect to a potential diagnosis as evidenced by one or more revised codes.

At optional step 118, one or more user interfaces are generated. The user interfaces may include a domain interface associated with one or more selected domains. For example, in the context of a medical domain, the domain interface may include a medical interface or patient interface. The one or more user interfaces may include one or more elements of support-based code structures 156 and/or one or more of the support-based next action recommendations 158. The user interfaces may provide outputs to users (e.g., individuals) based on received intake information. The outputs may include, but are not limited to, analysis of medical necessity for one or more tests, potential diagnosis, and/or any other suitable information. In some embodiments, a user interface includes an indication that outputs are provided for user information purposes only.

In some embodiments, one or more user interfaces are provided to users via an integrated network interface. The integrated network interface may provide a first interface, e.g., an information interface, to a first class of users, such as patients in the context of a medical domain, and a second interface, e.g., a next-action selection interface, to a second class of users, such as providers in the context of a medical domain. The information interfaces may provide the first class of users with information to be shared with the second class of users, such as during an in-office visit and/or via one or more electronic interactions. In some embodiments, the information interface enables sharing of one or more elements presented by the information interface, such as one or more support-based code structures 156 and/or one or more of the support-based next action recommendations 158, to one or more selected users in the second class of users (e.g., a patient can choose to share certain information with one or more providers via the information interface). In some embodiments, the information interface may provide additionally outputs, such as potential products or services that may be beneficial based on the support-based code structures 156 and/or one or more of the support-based next action recommendations 158.

In some embodiments, an information interface may include a first portion of the support-based code structures 156 and/or one or more of the support-based next action recommendations 158 identified based on received intake data 152 and a next-action selection interface may include a second portion of the support-based code structures 156 and/or one or more of the support-based next action recommendations 158. In some embodiments, the next-action selection interface may include all of the first portion of information presented via the information interface and additional information not presented via the information interface.

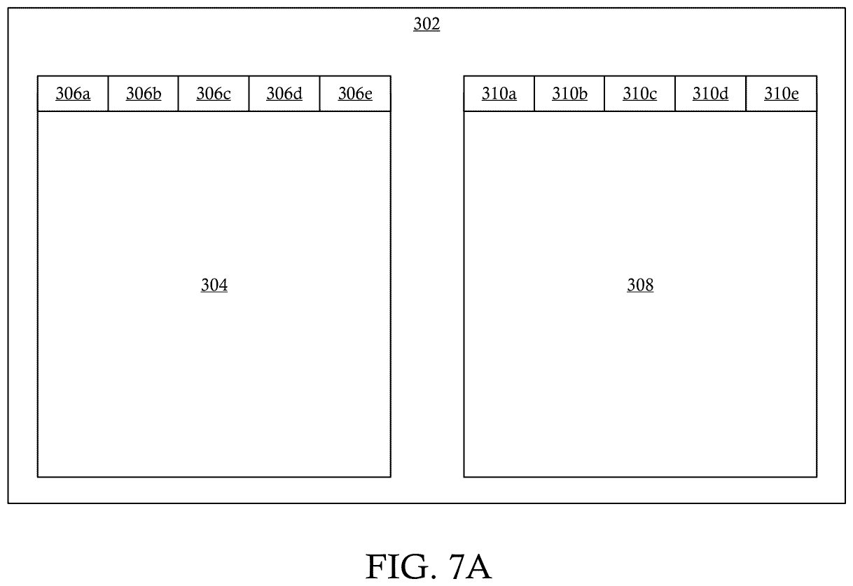

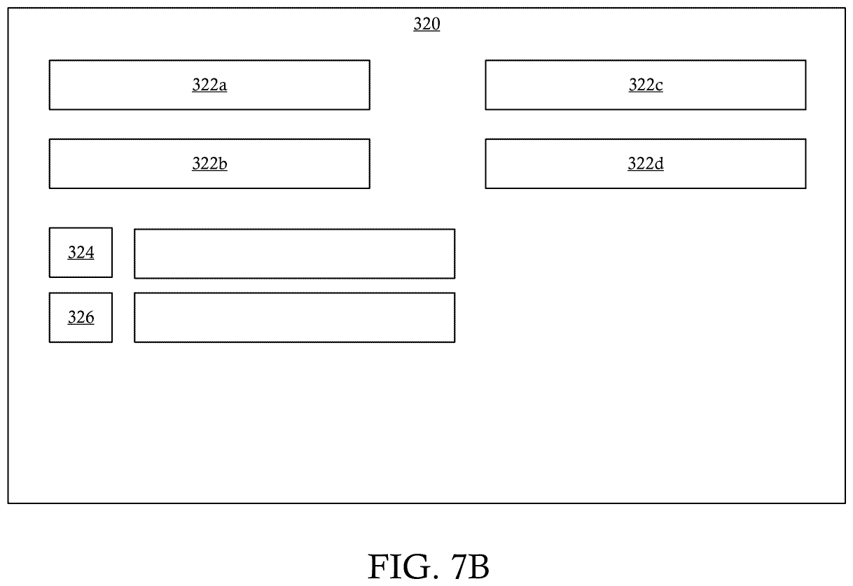

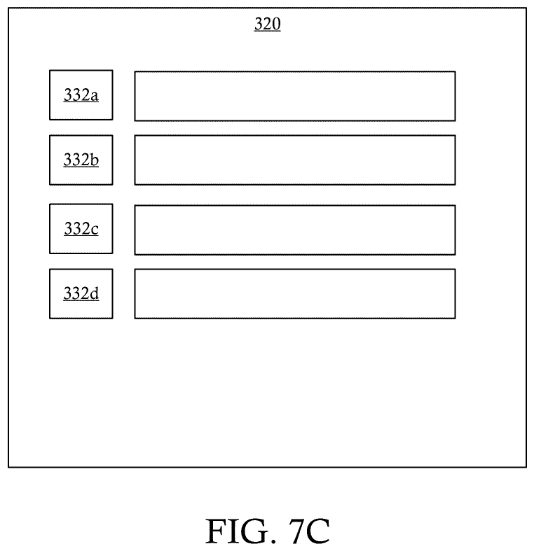

FIGS. 7A-7C illustrate various interfaces for presenting and/or interacting with elements of the disclosed systems and methods, in accordance with some embodiments. FIG. 7A illustrates a domain support interface 302 associated with a medical domain. The domain support interface 302 is configured to display portions of one or more support-based identified code structures 256, such as, evidenced codes 258, support elements 260, generated information 264, return data 168, and/or any other suitable data. In the illustrated embodiment, the domain support interface 302 includes a first portion 304 configured to display intake data and/or domain codex data (e.g., medical codex data) and a second portion 308 configured to display generated natural language information.

As illustrated in FIG. 7A, in some embodiments including a medical domain, the first portion 304 of the interface 302 may include medical codex data tabs 306a-306e. The medical codex data tabs 306a-306e may include, but are not limited to, patient details, patient history, potential diagnosis, recommended next steps, lab reports, body metrics, etc. Each medical codex data tab 306a-306e may be configured to display a portion of received intake data 152, evidenced codes 258, support elements 260, generated information 264, return data 168, etc. The data displayed may be organized according to one or more categorizations and/or groupings.

In some embodiments, at least one of the medical codex data tabs 306a-306e includes data related to one or more of the evidenced codes 258. For example, a medical codex data tab 306c may include each of the support-based evidenced codes 258 and support elements 260. In some embodiments, the support-based evidenced codes 258 may be displayed in conjunction with one or more support-based next-action recommendations 158 corresponding to the evidenced code 258. For example, a support-based evidenced code 258 may be displayed with multiple recommended tests that may be executed to verify and/or reject the evidenced code 258 and/or the corresponding diagnosis.

Similarly, as further illustrated in FIG. 7A, in some embodiments, the second portion 308 includes content tabs 310a-310e. The content tabs 310a-310e may include, but are not limited to, possible diagnosis (e.g., potential codes), treatment protocols, medication, lifestyle changes, sample conversations, etc. Each of the content tabs 310a-310e may include content, such as textual content, generated by one or more generative models. Each content tab 310a-310e may include content generated using modified prompts and/or inputs to a generative model to provide sub-domain, or topic-specific, generated information. In some embodiments, generated content may include content generated by providing a prompt including a potential code and/or a specific persona or scenario to a generative model. As one non-limiting example, a generative model may be configured to generate one or more sample conversations based on a corresponding potential code and a domain-specific persona or scenario, such as, for example, providing an ICD-10 code related to a cardiovascular diagnoses and a persona definition identifying the generative model as a cardiologist and a scenario identifying a patient conversation with the cardiologist regarding the diagnosis embodied in the potential code.

FIG. 7B illustrates an intake form 320 configured to receive intake data in one or more fields and/or forms. The intake form 320 may include any suitable data entry element, such as, for example, fillable fields 322a-322d, check boxes 324, radio buttons 326, drop down menus, etc. The intake form 320 may be configured to obtain domain-specific information required by and/or significant for the identification of one or more evidenced codes 258.

FIG. 7C illustrates a provider dashboard form 330 configured to allow a provider to authorize generation of intake datasets, such as intake data 352, from one or more outside providers. The provider dashboard 330 may enable a user to allow additional users to access portions of the disclosed systems and methods, such as, for example, to interact with one or more elements generated during, for example, data intake and next-action recommendation method 100.

Identification of codes within a codec based on relevant support, actions to be taken based on identified codes, and generation of content based on identified codes and/or recommended actions can be burdensome and time consuming for users, especially where such processes are not supported by automated systems. Typically, a user may locate information regarding certain codes or actions by navigating a browse structure, sometimes referred to as a “browse tree,” in which interface pages or elements are arranged in a predetermined hierarchy. Such browse trees typically include multiple hierarchical levels, requiring users to navigate through several levels of browse nodes or pages to arrive at an interface page of interest. Thus, the user frequently has to perform numerous navigational steps to find corresponding codes and must rely on large volumes of institutional knowledge in order to properly navigate corresponding browse trees.

Systems including automated identification of support-based codes and/or recommended next actions, as disclosed herein, significantly reduce this problem, allowing such codes and actions to be identified with fewer, or in some case no, active steps. For example, in some embodiments described herein, Beneficially, programmatically identifying support-based codes and actions and presenting a user with navigations shortcuts to these tasks may improve the speed of the user's navigation through an electronic interface, rather than requiring the user to page through multiple other pages or navigate obtuse browse trees.

It will be appreciated that identification of support-based evidenced codes and/or support-based next-action recommendations as disclosed herein, particularly on large datasets intended to be used within a medical domain during an intake process, is only possible with the aid of computer-assisted machine-learning algorithms and techniques, such as the disclosed classification module and/or generative models. In some embodiments, machine learning processes including classification modules and/or generative models are used to perform operations that cannot practically be performed by a human, either mentally or with assistance, such as identification of support-based evidenced codes and/or support-based next-action recommendations.

Although the subject matter has been described in terms of exemplary embodiments, it is not limited thereto. Rather, the appended claims should be construed broadly, to include other variants and embodiments, which may be made by those skilled in the art.

What is claimed is:

1. A system, comprising:
a processor; and
a non-transitory memory storing instructions that, when executed, cause the processor to:
receive a set of intake data;
generate at least one support-based evidenced code based on the set of intake data;
identify at least one support-based recommended action for the at least one support-based evidenced code;
receive a selection of the at least one support-based recommended action; and
transmit instructions to cause execution of the at least one support based recommended action.

2. The system of claim 1, wherein the at least one support-based evidenced code is generated by:
generating a set of evidenced code classifications; and
selecting the at least one support-based evidenced code from the set of evidenced code based on two or more dimensions of the intake data.

3. The system of claim 2, wherein the at least one support-based evidenced code is selected by a classification model that generates a confidence score, and wherein the confidence score of the at least one support-based evidenced code is above a predetermined threshold.

4. The system of claim 2, wherein the at least one support-based evidenced code is selected by a spectrum analysis.

5. The system of claim 1, wherein the at least one support-based evidence code is included in a support-based code structure including the at least one support-based evidence code and at least one support element.

6. The system of claim 5, wherein the support-based code structure includes at least one information element generated by a generative model.

7. The system of claim 6, wherein the generative model receives a prompt generated from at least a portion of the set of intake data and outputs the at least one information element in response to the prompt.

8. The system of claim 1, wherein the at least one support-based recommended action is selected from a set of domain-specific available actions.

9. A computer-implemented method, comprising:
receiving a set of intake data;
generating at least one support-based evidenced code based on the set of intake data;
identifying at least one support-based recommended action for the at least one support-based evidenced code;
receiving a selection of the at least one support-based recommended action; and
transmitting instructions to cause execution of the at least one support based recommended action.

10. The computer-implemented method of claim 9, wherein the at least one support-based evidenced code is generated by:
generating a set of evidenced code classifications; and
selecting the at least one support-based evidenced code from the set of evidenced code based on two or more dimensions of the intake data.

11. The computer-implemented method of claim 10, wherein the at least one support-based evidenced code is selected by a classification model that generates a confidence score, and wherein the confidence score of the at least one support-based evidenced code is above a predetermined threshold.

12. The computer-implemented method of claim 10, wherein the at least one support-based evidenced code is selected by a spectrum analysis.

13. The computer-implemented method of claim 9, wherein the at least one support-based evidence code is included in a support-based code structure including the at least one support-based evidence code and at least one support element.

14. The computer-implemented method of claim 13, wherein the support-based code structure includes at least one information element generated by a generative model.

15. The computer-implemented method of claim 14, wherein the generative model receives a prompt generated from at least a portion of the set of intake data and outputs the at least one information element in response to the prompt.

16. The computer-implemented method of claim 9, wherein the at least one support-based recommended action is selected from a set of domain-specific available actions.

17. A non-transitory computer-readable medium having instructions stored thereon, wherein the instructions, when executed by at least one processor, cause at least one device to perform operations comprising:
receiving a set of intake data;
generating at least one support-based evidenced code based on the set of intake data;
identifying at least one support-based recommended action for the at least one support-based evidenced code;
receiving a selection of the at least one support-based recommended action; and
transmitting instructions to cause execution of the at least one support based recommended action.

18. The non-transitory computer-readable medium of claim 17, wherein the instructions that cause generating of the at least one support-based evidenced code cause the at least one device to perform operations comprising:
generating a set of evidenced code classifications; and
selecting the at least one support-based evidenced code from the set of evidenced code based on two or more dimensions of the intake data.

19. The non-transitory computer-readable medium of claim 18, wherein the at least one support-based evidenced code is selected by a classification model that generates a confidence score, and wherein the confidence score of the at least one support-based evidenced code is above a predetermined threshold.

20. The non-transitory computer-readable medium of claim 18, wherein the at least one support-based evidenced code is selected by a spectrum analysis.
